SEQUENCE LISTING (32 sequences)

SEQ ID NO: 1 - DNA, 867 bp, Homo sapiens
  CDS (1)..(867)
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctga

SEQ ID NO: 2 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 3 - DNA, 531 bp, Homo sapiens
  CDS (1)..(531)
atgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 4 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 5 - DNA, 822 bp, Homo sapiens
  CDS (1)..(822)
atgatcttcctcctgctaatgttgagcctggaattgcagcttcaccagatagcagctttattcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaagtcaaagcttcctacaggaaaataaacactcacatcctaaaggttccagaaacagatgaggtagagctcacctgccaggctacaggttatcctctggcagaagtatcctggccaaacgtcagcgttcctgccaacaccagccactccaggacccctgaaggcctctaccaggtcaccagtgttctgcgcctaaagccaccccctggcagaaacttcagctgtgtgttctggaatactcacgtgagggaacttactttggccagcattgaccttcaaagtcagatggaacccaggacccatccaacttggctgcttcacattttcatccccttctgcatcattgctttcattttcatagccacagtgatagccctaagaaaacaactctgtcaaaagctgtattcttcaaaagacacaacaaaaagacctgtcaccacaacaaagagggaagtgaacagtgctatctga

SEQ ID NO: 6 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 7 - DNA, 525 bp, Homo sapiens
  CDS (1)..(525)
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgctaaagaaaagaagccctcttacaacaggggtctatgtgaaaatgcccccaacagagccagaatgtga

SEQ ID NO: 8 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 9 - DNA, 867 bp, Homo sapiens
  CDS (1)..(867)
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataa

SEQ ID NO: 10 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 11 - DNA, 654 bp, Homo sapiens
  CDS (1)..(654)
atggatccccagtgcactatgggactgagtaacattctctttgtgatggccttcctgctctctgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaa

SEQ ID NO: 12 - protein, 217 aa, Homo sapiens
MDPQCTMGLSNILFVMAFLLSANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 13 - DNA, 1605 bp, Homo sapiens
  CDS (1)..(1605)
atgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggccctgtgggtgaccgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctga

SEQ ID NO: 14 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 15 - DNA, 564 bp, Homo sapiens
  CDS (1)..(564)
atgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaataa

SEQ ID NO: 16 - protein, 187 aa, Homo sapiens
MFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 17 - DNA, 936 bp, Homo sapiens
  CDS (1)..(936)
atgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctag

SEQ ID NO: 18 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 19 - DNA, 1335 bp, Homo sapiens
  CDS (1)..(1335)
atgttgctcatggtcgtcagcatggcgtgtgttgggttcttcttggtccagagggccggtccacacgtgggtggtcaggacaagcccttcctgtctgcctggcccagcgctgtggtgcctcgaggaggacacgtgactcttcggtgtcactatcgtcataggtttaacaatttcatgctatacaaagaagacagaatccacgttcccatcttccatggcagattattccaggagagcttcaacatgagccctgtgaccacagcacatgcagggaactacacatgtcggggttcacacccacactcccccactgggtggtcggcacccagcaaccccgtggtgatcatggtcacaggaaaccacagaaaaccttccctcctggcccacccaggtcccctggtgaaatcaggagagagagtcatcctgcaatgttggtcagatatcatgtttgagcacttctttctgcacaaagaggggatctctaaggacccctcacgcctcgttggacagatccatgatggggtctccaaggccaatttctccatcggtcccatgatgcttgcccttgcagggacctacagatgctacggttctgttactcacaccccctatcagttgtcagctcccagtgatcccctggacatcgtggtcacaggtccatatgagaaaccttctctctcagcccagccgggccccaaggttcaggcaggagagagcgtgaccttgtcctgcagctcccggagctcctatgacatgtaccatctatccagggaggggggagcccatgaacgtaggctccctgcagtgcgcaaggtcaacagaacattccaggcagatttccctctgggccctgccacccacggagggacctacagatgcttcggctctttccgtcactctccctacgagtggtcagacccgagtgacccactgcttgtttctgtcacaggaaacccttcaagtagttggccttcacccacagaaccaagctccaaatctggtaaccccagacacctgcacattctgattgggacctcagtggtcatcatcctcttcatcctcctcctcttctttctccttcatctctggtgctccaacaaaaaaaatgctgctgtaatggaccaagagcctgcagggaacagaacagccaacagcgaggactctgatgaacaagaccctgaggaggtgacatacgcacagttggatcactgcgttttcacacagagaaaaatcactcgcccttctcagaggcccaagacaccccctacagataccatcttgtacacggaacttccaaatgctaagcccagatccaaagttgtctcctgcccatga

SEQ ID NO: 20 - protein, 444 aa, Homo sapiens
MLLMVVSMACVGFFLVQRAGPHVGGQDKPFLSAWPSAVVPRGGHVTLRCHYRHRFNNFMLYKEDRIHVPIFHGRLFQESFNMSPVTTAHAGNYTCRGSHPHSPTGWSAPSNPVVIMVTGNHRKPSLLAHPGPLVKSGERVILQCWSDIMFEHFFLHKEGISKDPSRLVGQIHDGVSKANFSIGPMMLALAGTYRCYGSVTHTPYQLSAPSDPLDIVVTGPYEKPSLSAQPGPKVQAGESVTLSCSSRSSYDMYHLSREGGAHERRLPAVRKVNRTFQADFPLGPATHGGTYRCFGSFRHSPYEWSDPSDPLLVSVTGNPSSSWPSPTEPSSKSGNPRHLHILIGTSVVIILFILLLFFLLHLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 21 - DNA, 768 bp, Homo sapiens
  CDS (1)..(768)
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 22 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 23 - DNA, 1578 bp, Homo sapiens
  CDS (1)..(1578)
atgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcctgctgtttctcatccttggtgtcctttctctgctccttttggtgactggagcctttggctttcacctttggagaagacagtggcgaccaagacgattttctgccttagagcaagggattcaccctccgcaggctcagagcaagatagaggagctggagcaagaaccggagccggagccggagccggaaccggagcccgagcccgagcccgagccggagcagctctga

SEQ ID NO: 24 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 25 - DNA, 906 bp, Homo sapiens
  CDS (1)..(906)
atgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactcggcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatag

SEQ ID NO: 26 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 27 - DNA, 735 bp, Homo sapiens
  CDS (1)..(735)
atgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttag

SEQ ID NO: 28 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 29 - DNA, 834 bp, Homo sapiens
  CDS (1)..(834)
atgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatctga

SEQ ID NO: 30 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 31 - DNA, 726 bp, Homo sapiens
  CDS (1)..(726)
atgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggaccgaactcaagactggcaagaaatgtaaaagaagcaccaacagaatatgcatccatatgtgtgaggagttaa

SEQ ID NO: 32 - protein, 241 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

(19) United States
(12) Patent Application Publication
(10) Pub. No.: US 2019/0358276 A1
(43) Pub. Date: Nov. 28, 2019

(54) NOVEL THERAPEUTIC AGENT FOR PRIONOID DISEASES

(71) Applicants: OSAKA UNIVERSITY, Suita (JP); FOUNDATION FOR BIOMEDICAL RESEARCH AND INNOVATION AT KOBE, Kobe (JP); GENOMIDEA INC., Osaka (JP)

(72) Inventors: Yasufumi KANEDA, Suita (JP); Masanori FUKUSHIMA, Kobe (JP); Toshihiro NAKAJIMA, Ikeda (JP)

(73) Assignees: OSAKA UNIVERSITY, Suita (JP); FOUNDATION FOR BIOMEDICAL RESEARCH AND INNOVATION AT KOBE, Kobe (JP); GENOMIDEA INC., Osaka (JP)

(21) Appl. No.: 16/466,206

(22) PCT Filed: Dec. 6, 2017
(86) PCT No.: PCT/JP2017/043725

§ 371 Date: Jun. 3, 2019

(30) Foreign Application Priority Data
Dec. 6, 2016 (JP) 2016-236536

(51) Int. Cl. A61K 35/76; A61P 25/28; C07K 16/28

(52) U.S. Cl. CPC A61K 35/76; C07K 16/2827; C07K 16/2818; A61P 25/28

(57) ABSTRACT

The present invention relates to a medicament for preventing and/or treating cognitive impairment and/or a neurodegenerative disease with accumulation of a prionoid, comprising a Sendai virus envelope as an active ingredient and combined application of the medicament and an immune checkpoint inhibitor.

### TECHNICAL FIELD

### Related Application

The Description of the present application encompasses the contents described in the Description of Japanese Patent Application No. 2016-236536 (filed on Dec. 6, 2016), to which the present application claims priority.

### Technical Field

The present invention relates to a medicament for treating cognitive impairment and/or a neurodegenerative disease, comprising a Sendai virus envelope as an active ingredient and combined application of the medicament and an immune checkpoint inhibitor.

### BACKGROUND ART

Dementia is a cognitive impairment that causes difficulties in daily life and social life due to chronicle decline or loss of normally developed mind functions by acquired structural disorder of the brain. There are several kinds of dementia such as Alzheimer-type dementia, vascular dementia, dementia with Lewy bodies, and frontotemporal dementia, but Alzheimer-type dementia among others is most common, being said to account for 60% of the causes of dementia.

While neurodegeneration due to the accumulation of amyloid β is presumed to be most likely, there are various hypotheses about the cause of the Alzheimer-type dementia and the connection with the immune system is also suggested (Non Patent Literatures 1 and 2). Some medical treatments have also been developed for dementia and particularly Alzheimer-type dementia, but no radical treatment has been developed.

Sendai virus (Hemaglutinating virus of Japan (HVJ)) have attracted attention for causing fusion of Erich tumor cells. Its activity of fusing the cell membrane (hereinafter, referred to as the fusing activity) has been analyzed and the use of HVJ as a transfection vector has also been studied. However, HVJ has high immunogenicity and, when NP protein is produced in a large quantity, it is known to induce CTL in particular and there is also concern that the protein synthesis of the host is inhibited. Accordingly, a method for producing fused particles (HVJ-liposome) by fusing liposomes encapsulating a gene or a protein and HVJ inactivated by ultraviolet irradiation has been developed and this has made non-invasive transfection of cells and living bodies possible (Patent Literature 1, Non-Patent Literatures 3 and 4).

The present inventors have developed a novel hybrid transfection vector by combining a virus having high potency (high efficiency) of gene delivery and a nonviral vector having low cytotoxicity and immunogenicity (low toxicity) and constructed fusogenic viral liposome having a fusogenic envelope derived from HVJ. In this delivery system, DNA loaded liposome is fused with UV inactivated HVJ to form HVJ-liposome (400-500 nm in diameter), a fusogenic virus-liposome. The advantage of the fusion-mediated delivery is that transfected DNA is protected from the endosomal degradation and lysosomal degradation in acceptor cells. For example, DNA incorporated in HVJ-liposome can safely be delivered to mammalian cells (Patent Literature 2). Moreover, RNA, oligonucleotides, and drugs can efficiently be introduced into cells in vitro and in vivo. Furthermore, the present inventors have invented a transfection vector encapsulating an exogenous gene by freeze-thawing or mixing the HVJ envelope (HVJ-E) with a surfactant as a transfection vector having a virus envelope, being available for safely and stably transfecting a wide range of tissues in the living body, and having high transfection activity (Patent Literatures 3 and 4). This made it possible to efficiently introduce a substance into the brain or central nerves using HVJ-E (Patent Literatures 5 and 6). Furthermore, the inventors have found that HVJ-E exhibits immunological adjuvant effect by encapsulating a chemotherapeutic agent such as an anticancer agent into HVJ-E and transfecting the HVJ-E into cells or the living body (Patent Literatures 7 and 8).

The inventors have found that HVJ-E itself has the antitumor immunity-activating effect and the cancer cell-specific cell death-inducing effect and is useful as an anticancer agent (Patent Literatures 8 and 9) and have been conducting a doctor-led clinical trial for malignant melanoma patients, prostate cancer patients, and malignant pleural mesothelioma patients.

### CITATION LIST

### Patent Literature

Patent Literature 1: U.S. Pat. No. 5,631,237
Patent Literature 2: WO2001/057204
Patent Literature 3: Japanese Patent Laid-Open No. 2002-065278
Patent Literature 4: WO2004/035779
Patent Literature 5: WO2006/011600
Patent Literature 6: WO2005/095613
Patent Literature 7: WO2004/039406
Patent Literature 8: WO2005/094878
Patent Literature 9: WO2010/032764

### Non Patent Literature

Non Patent Literature 1: Schenk et al., Nature. 1999 Jul. 8, 400 (6740), p. 173-177
Non Patent Literature 2: Baruch et al., Nature Medicine, 2016 February vol. 22, No. 2, p. 135-137
Non Patent Literature 3: Dzau et al., PNAS 1996, Vol. 93, No. 21, p. 11421-11425
Non Patent Literature 4: Kaneda et al., Mol. Med. Today, 1999, Vol. 5, Issue 7, p. 298-303

### SUMMARY OF INVENTION

### Technical Problem

An object of the invention is to find a novel effect of HVJ-E and establish its clinical application.

### Solution to Problem

The inventors have found that administration of HVJ-E to model mice of Alzheimer's disease created by administration of 0 amyloid leads to significant improvement of short-term memory. Furthermore, it has been found that combined application of an anti-PD-1 antibody with HVJ-E leads to synergistic improvement of the effect.

The present invention has been completed based on the aforementioned findings and relates to the following (1) to (11).

(1) A medicament for preventing and/or treating cognitive impairment and/or a neurodegenerative disease with accumulation of a prionoid, comprising a Sendai virus envelope as an active ingredient (more specifically, in an effective dose).
(2) The medicament according to (1), wherein the Sendai virus envelope is a wild type Sendai virus envelope.
(3) The medicament according to (1) or (2), wherein the Sendai virus envelope is an inactivated Sendai virus envelope.
(4) The medicament according to any of (1) to (3), wherein the cognitive impairment and/or a neurodegenerative disease with accumulation of a prionoid is any one selected from Alzheimer-type dementia, mild cognitive impairment (MCI), cerebral amyloid angiopathy, Down syndrome, macular degeneration, dementia with Lewy bodies, Parkinson's disease, multiple system atrophy, tauopathy, frontotemporal lobar degeneration, argyrophilic grain dementia, amyotrophic lateral sclerosis, autism, diabetes, amyotrophic lateral sclerosis (ALS), and Creutzfeldt-Jakob disease.
(5) The medicament according to (4), wherein the cognitive impairment and/or a neurodegenerative disease with accumulation of a prionoid is Alzheimer-type dementia.
(6) The medicament according to any of (1) to (5), wherein the prionoid is amyloid β.
(7) The medicament according to any of (1) to (6), wherein the medicament is administered in combination with an immune checkpoint inhibitor.
(8) The medicament according to (7), wherein the Sendai virus envelope and the immune checkpoint inhibitor are administered simultaneously or sequentially.
(9) The medicament according to any of (1) to (8), comprising the Sendai virus envelope and the immune checkpoint inhibitor in combination.
(10) The medicament according to any of (7) to (9), wherein the immune checkpoint inhibitor is any one or more selected from the group consisting of an anti-PD-1 antibody, an anti-PD-L1 antibody, an anti-PD-L2 antibody, an anti-CTLA-4 antibody, an anti-KIR antibody, an anti-CD137 antibody, an anti-LAG-3 antibody, an anti-OX40 antibody, an anti-CD80 antibody, an anti-CD86 antibody, an anti-B7-H3 antibody, an anti-B7-H4 antibody, an anti-B7-H5 antibody, an anti-TIM-3 antibody, an anti-TIGIT antibody, and an anti-BTLA antibody.
(11) The medicament according to (10), wherein the immune checkpoint inhibitor is an anti-PD-1 antibody, an anti-PD-L1 antibody, or an anti-CTLA-4 antibody and preferably an anti-PD-1 antibody.

### Advantageous Effects of Invention

The present invention provides a new therapeutic method for treating and/or preventing cognitive impairment including Alzheimer-type dementia and/or a neurodegenerative disease. Cognitive impairment or a neurodegenerative disease with accumulation of a prionoid such as β amyloid, for example, Alzheimer-type dementia, can be treated with HVJ-E more effectively by a combined application with an immune checkpoint inhibitor. The combination effect of HVJ-E and an immune checkpoint inhibitor is synergistic and therefore the combined application can decrease the doses of the agents in comparison with single administration and reduce side effects.

### BRIEF DESCRIPTION OF DRAWINGS

FIG. 1 illustrates an apparatus for the Y-maze test and a method for calculating the spontaneous alternation rate. In the figure, as illustrated in the calculation example, if an animal moves in the order of ACBABACBAB, when the arms of the Y-shaped maze are referred to as A, B, and C, then the number of spontaneous alternation is 5 of ACB, CBA, BAC, ACB, CBA and this is divided by 8, which is the number obtained by subtracting 2 from the total number of entries 10, and multiplied by 100 to obtain a spontaneous alternation rate of 62.5. The spontaneous alternation rate (%)=[5/(10−2)]×100=62.5%

FIG. 2 illustrates the result (HVJ-E is administered subcutaneously) of the Y-maze test. FIG. 2A: the number of total entries, FIG. 2B: the number of spontaneous alternation (the number of correct answers), and FIG. 2C: the spontaneous alternation rate (the rate of correct answer). From the left in all the panels, the negative control group (vehicle administered group), the HVJ-E administered group, the anti-PD-1 antibody administered group, the HVJ-E+ anti-PD-1 antibody combined application group, and the positive control group (donepezil administered group).

FIG. 3 illustrates the result (HVJ-E is administered subcutaneously) of the passive test (reaction latency). FIG. 3A: Day 1 (Day 10 after the administration of the test substance), FIG. 3B: Day 2 (Day 11 after the administration of the test substance). From the left in all the panels, the negative control group (vehicle administered group), the HVJ-E administered group, the anti-PD-1 antibody administered group, the HVJ-E+ anti-PD-1 antibody combined application group, and the positive control group (donepezil administered group).

FIG. 4 illustrates the result of the Y-maze test (HVJ-E is administered intranasally). FIG. 4A: the number of total entries, FIG. 4B: the number of spontaneous alternation (the number of correct answers), and FIG. 4C: the spontaneous alternation rate (the rate of correct answer). From the left in all the panels, the negative control group (vehicle administered group), the HVJ-E intranasally administered group, the HVJ-E (intranasal)+ anti-PD-1 antibody combined application group, the positive control group (donepezil administered group), and the HVJ-E (subcutaneous)+ anti-PD-1 antibody combined application group. In FIG. 4, Sham-operation indicates a sham operation group, Vehicle indicates the vehicle administered group, i.n. indicates intranasal administration, and s.c. indicates subcutaneous administration.

FIG. 5 illustrates the result of the Y-maze test (HVJ-E is administered intranasally). FIG. 5A: the number of total entries, FIG. 5B: the number of spontaneous alternation (the number of correct answers), and FIG. 5C: the spontaneous alternation rate (the rate of correct answer). From the left in all the panels, the negative control group (vehicle administered group), the HVJ-E (10 mNAU/body) nasal administered group, the HVJ-E (50 mNAU/body) nasal administered group, the HVJ-E (100 mNAU/body) nasal administered group, and the positive control group (donepezil administered group). In FIG. 5, Vehicle indicates the vehicle administered group.

### DESCRIPTION OF EMBODIMENTS

The present invention relates to a medicament for preventing and/or treating cognitive impairment and/or a neurodegenerative disease with accumulation of a prionoid, comprising a Sendai virus envelope (HVJ-E) as an active ingredient.

### 1. Medicament Comprising Sendai Virus Envelope

### “Sendai Virus (HVJ)”

The “Sendai virus (HVJ)” is a single strand minus strand RNA virus in the genus Respirovirus of the family Paramyxoviridae, infects mice and rats, and causes a respiratory disease. On the outer side of the virus particle, an envelope, which is a membranous structure, exists, and covers the virus genome and capsid proteins. The envelope proteins are membrane components responsible for the cell fusion activity when the virus adheres to and invades a host cell and play an important role in viral infection, such as the immune evasion.

### “Sendai Virus Envelope (HVJ-E)”

The “Sendai virus envelope (HVJ-E)” according to the present invention is an inactivating Sendai virus which has been inactivated and lost the replication competence (Kaneda et al., Hemagglutinating Virus of Japan (HVJ) Envelope Vector as a Versatile Gene Delivery System. Mol. Ther. 2002, 6, 219-226). HVJ-E does not have the infectivity and the proliferative capacity in the host since the viral genomic RNA has been completely inactivated,

The Hemagglutinin-Neuraminidase (HN) protein and the Fusion (F) protein, which are proteins on the membrane of HVJ-E, maintain the cell membrane fusion ability even after the viral RNA inactivation. Therefore, it is possible to encapsulate a gene or a drug in the inactivated HVJ envelope and deliver to cells of interest. Moreover, HVJ-E can be used for the treatment of cancer alone or encapsulating an anticancer agent since it has the adjuvant effect of increasing tumor immunity (mentioned above, WO2005/094878, WO2010/032764). It has been not known before the present application that HVJ-E has the preventive and therapeutic effect on cognitive impairment and neurodegenerative diseases with the accumulation of a prionoid such as Alzheimer's disease.

### “Preparation of HVJ-E”

The HVJ-E according to the present invention can be obtained by inactivating HVJ. The HVJ to be used may be the wild type or a mutant with an appropriate modification as long as it does not cause deteriorating effect on the purpose as a medicament. Examples of the wild type HVJ that can be used include commercially available ATCC® VR-105™, ATCC® VR-907™, and the like. The virus is preferably purified prior to the inactivation by a combination of centrifugation or ultrafiltration and an ion exchange column.

Examples of the mutant HVJ include HVJ-E in which HN has been deleted by using an siRNA specific to the mRNA or the like; HVJ-E modified to express a fusion protein of a modified HN protein and a desired protein on the outer surface of the HVJ-E by connecting a desired polypeptide to the C-terminal of the particular region of the HN protein composing the HVJ-E (see Japanese Patent Laid-Open No. 2011-050292); other HVJ-Es in which a membrane protein is modified, and the like.

The inactivation of the virus is not particularly limited as long as it eliminates the replication competence of the virus in host cells, preferably human cells and may be carried out by ultraviolet irradiation, gamma ray irradiation, chemical treatment (alkylating agent treatment), heat-treatment, or the like, but ultraviolet irradiation and gamma beam irradiation are preferred from the viewpoint that the effect on proteins on the envelope membrane is small. In the case of ultraviolet irradiation and gamma ray irradiation, the virus can be inactivated by irradiating a suspension of HVJ with an ultraviolet ray (usually around 90 to 200 millijoules/cm²) or a gamma ray (usually around 5 to 20 grays). Moreover, in the case of the alkylating agent treatment, the virus may be inactivated by adding an alkylating agent such as β propiolactone to a suspension of the HVJ and incubating the suspension. Specific methods for preparing HVJ-E are described in detail in Kaneda et al., 2002, WO01/57204 (Example 8), Japanese Patent Laid-Open No. 2002-065278, and WO03/014338 mentioned above.

### “Medicament Comprising HVJ-E”

The medicament comprising HVJ-E according to the present invention may comprise a pharmacologically acceptable carrier and/or an additive. Examples of such a carrier and an additive include a filler, a binder, a lubricant, a solvent, a disintegrator, a solubilizing agent, a suspending agent, an emulsifier, an isotonizing agent, a stabilizer, a soothing agent, an antiseptic, an antioxidant, a corrigent, a colorant, a buffer, a superplasticizer, and the like, but are not limited thereto and other commonly used carriers and/or additives may be used.

Specifically, examples of the filler include organic fillers such as saccharides such as lactose, glucose, and D-mannitol, starches, and cellulose such as crystalline cellulose; inorganic fillers such as calcium carbonate and kaolin; and the like.

Examples of the binder include pregelatinized starch, gelatin, Arabian gum, methylcellulose, carboxymethylcellulose, sodium carboxymethylcellulose, microcrystalline cellulose, D-mannitol, trehalose, hydroxypropylcellulose, hydroxypropylmethylcellulose, polyvinylpyrrolidone, polyvinyl alcohol, and the like.

Examples of the lubricant include stearic acid, fatty acid salts such as stearates, talc, silicates, and the like.

Examples of the solvent include purified water, physiological saline solutions, phosphate buffer solutions, and the like.

Examples of the disintegrator include low-substituted hydroxypropyl cellulose, chemically modified cellulose and starches, and the like.

Examples of the solubilizing agent include polyethyleneglycol, propylene glycol, trehalose, benzyl benzoate, ethanol, sodium carbonate, sodium citrate, sodium salicylate, sodium acetate, and the like.

Examples of the suspending agent or emulsifier include sodium lauryl sulfate, Arabian gum, gelatin, lecithin, glyceryl monostearate, polyvinyl alcohol, polyvinylpyrrolidone, cellulose such as sodium carboxymethylcellulose, polysorbate, polyoxyethylene hydrogenated castor oils, and the like.

Examples of the isotonizing agent include sodium chloride, potassium chloride, saccharide, glycerin, urea, and the like.

Examples of the stabilizer include polyethyleneglycol, dextran sodium sulfate, amino acids, and the like.

Examples of the soothing agent include glucose, calcium gluconate, procaine hydrochloride, and the like.

Examples of the antiseptic include p-hydroxybenzoates, chlorobutanol, benzyl alcohol, phenethyl alcohol, dehydroacetic acid, sorbic acid, and the like.

Examples of the antioxidant include water-soluble antioxidants such as ascorbic acid, cysteine hydrochloride, sodium bisulfate, sodium metabisulfite, and sodium sulfite; liposoluble antioxidants such as ascorbyl palmitate, butylated hydroxyanisole (BHA), butylated hydroxytoluene (BHT), lecithin, propyl gallate, and α-tocopherol; and metal chelating agents such as citric acid, ethylenediaminetetraacetic acid (EDTA), sorbitol, tartaric acid, and phosphoric acid.

Examples of the corrigent include sweeteners and flavors commonly used in the field of medicament, and the like and examples of the colorant include coloring agents commonly used in the field of medicament.

HVJ-E may be formulated with a polymer such as hyaluronic acid, polylactic acid, alginic acid, and gelatin and/or a cationization agent such as cationized gelatin (Japanese Patent Laid-Open No. 2008-308,440), besides others.

The route of administration of the medicament according to the present invention is not particularly limited and the administration may be oral or parenteral administration. Specific examples of the parenteral administration include injection, transnasal administration, pulmonary administration, transdermal administration, and the like. Examples of the injection include intravenous injection, intramuscular injection, intraperitoneal injection, subcutaneous injection, intraosseous injection, intraspinal injection, and intradermal injection. The mode of administration can be selected by the age and symptoms of the patient.

The dose of the medicament according to the present invention can determined depending on the purpose of its use and the route of administration. When administered to humans, a dose, for example, in the range of 200 mNAU to 20,000 mNAU in terms of HVJ-E per dosing may be selected. Alternatively, a dose for example, in the range of 3,000 to 60,000 mNAU/body per patient may be selected.

The medicament according to the present invention may be administered in combination with another agent as long as it does not cause deteriorating effect on the purpose of the present invention. As described above, a nucleic acid or an agent may be encapsulated in HVJ-E. Also, in the present invention, an agent or the like useful for the treatment of cognitive impairment or a neurodegenerative disease may be encapsulated in HVJ-E as long as it does not cause deteriorating effect on the purpose of the present invention.

“Cognitive Impairment and/or Neurodegenerative Disease”

The medicament comprising HVJ-E is useful for the treatment and/or prophylaxis of cognitive impairment with the accumulation of a prionoid and/or a neurodegenerative disease with the accumulation of a prionoid.

The “prionoid” is a protein that is considered to cause the expansion of a lesion(s) by the transmission of a released missfolded protein to neighboring cells, and examples of the prionoid include amyloid β, α-synuclein, huntingtin, phosphorylated tau, and the like. Representative examples of the prionoid are “amyloid β” and “α-synuclein” described below.

The “amyloid β-” is a peptide that is a key component of the senile plaque, which is a main pathological change in Alzheimer-type dementia. The amyloid β (hereinafter, also referred to as “Aβ”) is a highly hydrophobic peptide that is produced by cutting out by β- and γ-secretase from the precursor protein (APP: amyloid β protein precursor) and includes Aβ₁₋₄₀ consisting of 40 amino acids and Aβ₁₋₄₂ consisting of 42 amino acids. Aβ₁₋₄₂ is more fibrillogenic than Aβ₁₋₄₀ and a hypothesis that Aβ₁₋₄₂ forms aggregates and then Aβ₁₋₄₀ aggregates on the Aβ₁₋₄₂ aggregates core to form fibrils has been proposed. However, it is considered that the modification by another risk factor(s) (such as apolipoprotein E and presenilin 1.2) is necessary for the accumulation of amyloid β. Aβ has the nerve cell toxicity and causes necrosis and the like.

The “α-synuclein” is a protein having unknown functions that is mainly found in neural tissues and consists of 140 amino acid residues encoded by the SNCA gene. The α-synuclein was first discovered as a component in the amyloid whose fragments are accumulated in Alzheimer disease, but it is considered to be a cause of neurodegenerative diseases including Parkinson disease.

Examples of the “cognitive impairment and/or a neurodegenerative disease with accumulation of a prionoid” include, but are not limited to, Alzheimer-type dementia, mild cognitive impairment (MCI), cerebral amyloid angiopathy, Down syndrome, macular degeneration, dementia with Lewy bodies, Parkinson's disease, multiple system atrophy, tauopathy, frontotemporal lobar degeneration, argyrophilic grain dementia, amyotrophic lateral sclerosis, autism, diabetes, amyotrophic lateral sclerosis (ALS), and Creutzfeldt-Jakob disease, and the like.

In the present invention, the term “treatment” means that various symptoms caused by cognitive impairment and/or a neurodegenerative disease with accumulation of a prionoid are improved by administering the medicament according to the present invention to a subject. Moreover, the word “prophylaxis” means that the onset and exacerbation of the cognitive impairment and/or neurodegenerative disease with accumulation of a prionoid is prevented.

### 2. Combination of HVJ-E and Immune Checkpoint Inhibitor

The inventors have confirmed that administration of an immune checkpoint inhibitor to model mice of Alzheimer's disease leads to a behavioral improvement and also demonstrated that the effect thereof exhibits synergistic effect by a combined application with HVJ-E. More specifically, the present invention also provides a medicament for preventing and/or treating cognitive impairment and/or a neurodegenerative disease with accumulation of a prionoid by combining (using a combination of or mixing) a Sendai virus envelope (HVJ-E) and an immune checkpoint inhibitor.

### “Medicament Comprising HVJ-E and Immune Checkpoint Inhibitor in Combination”

In the present invention, the “medicament for preventing and/or treating cognitive impairment and/or a neurodegenerative disease with accumulation of a prionoid, comprising HVJ-E and an immune checkpoint inhibitor in combination” means a medicament in which HVJ-E and an immune checkpoint inhibitor are combined for administering them simultaneously, separately, or sequentially in prophylaxis and/or treatment of a neurodegenerative disease. In one embodiment, the medicament according to the present invention is provided in a form of a combination drug comprising HVJ-E and an immune checkpoint inhibitor together. Moreover, in another embodiment, the medicament according to the present invention is provided as a combined application of separate agents comprising HVJ-E and an immune checkpoint inhibitor and the agents are used simultaneously or sequentially. Furthermore, in another embodiment, the medicament according to the present invention may be provided as a kit composed of an agent(s) comprising HVJ-E and an immune checkpoint inhibitor.

In the present invention, the “combined application” of HVJ-E and an immune checkpoint inhibitor means administration or use of HVJ-E and an immune checkpoint inhibitor in combination and the order or intervals of the administration thereof is not limited. Moreover, the “combination drug” of HVJ-E and an immune checkpoint inhibitor means a medicament formulated by combining certain amounts of HVJ-E and an immune checkpoint inhibitor (fixed dose combination drug).

The combined administration (use) of HVJ-E and an immune checkpoint inhibitor allows lower doses than the doses in use of either of the drugs alone and therefore can reduce the risk of side effects.

### “Immune Checkpoint Inhibitor”

The “immune checkpoint inhibitor” means to inhibit the immune checkpoint and inhibit the effect of substances and/or molecules that suppress the defense system of acquired immunity. The immune checkpoint is a molecule that suppresses the defense system of acquired immunity to pathogens such as cancer cells, bacteria, and viruses and examples thereof include PD-1, which is expressed on the surface of effector T cells, PD-L1 and PD-L2, which are expressed on the surface of tumor cells, CTLA-4, which is expressed on the surface of activated T cells or the suppressor T cell Treg, and CD80 (B7-1) and CD86 (B7-2), which are expressed on the surface of dendritic cells, as well as B7-H3, B7-H4, B7-H5 (VISTA), KIR, CD137, LAG-3, TIM-3, TIGIT, OX40, BTLA, and the like.

The amino acid sequences of the aforementioned immune checkpoint inhibitors and the nucleotide sequences of the genes encoding them are already known and disclosed in public databases (the nucleic acid sequences: GenBank, DDBJ, and EMBL, the amino acid sequences: SwissProt, PIR, and PDB). For example, the information of the gene of human PD-1 (Homo sapiens programmed cell death 1 (PDCD1), mRNA) is disclosed as GenBank Accession No. NM_005018 and the information of the protein thereof (programmed cell death protein 1 precursor [Homo sapiens]) is disclosed as Accession No. NP 005009 (https://www.ncbi.nlm.nih.gov/nuccore/NM_005018.2 and https://www.ncbi.nlm.nih.gov/protein/167857792). The immune checkpoint inhibitors (for example, antisense nucleic acids, siRNA, shRNA, and antibodies) described below can be designed and obtained based on the disclosed information using a technology known in the field. The information about the immune checkpoint inhibitors are shown together in the following.

TABLE 1

| | | Nucleotide | Amino acid |
|---|---|---|---|
| Immun-Checkpoint | mRNA, GenBank Accession No. | sequence (cDNA) | sequence |
| hPD-1 | NM_005018.2 (Gene ID: 5133) | SEQ ID NO: 1 | SEQ ID NO: 2 |
| hPD-L1 | NM_001267706.1 (Gene ID: 29126) | SEQ ID NO: 3 | SEQ ID NO: 4 |
| hPD-L2 | NM_025239.3 (Gene ID: 80380) | SEQ ID NO: 5 | SEQ ID NO: 6 |
| hCTLA-4 | NM_001037631.2 (Gene ID: 1493) | SEQ ID NO: 7 | SEQ ID NO: 8 |
| hCD80(B7-1) | NM_005191.3 (Gene ID: 941) | SEQ ID NO: 9 | SEQ ID NO: 10 |
| hB7.2(B7-2) | NM_001206924.1 (Gene ID: 942) | SEQ ID NO: 11 | SEQ ID NO: 12 |
| hB7-H3 | NM_001024736.1 (Gene ID: 80381) | SEQ ID NO: 13 | SEQ ID NO: 14 |
| hB7-H4 | NM_001253849.1 (Gene ID: 79679) | SEQ ID NO: 15 | SEQ ID NO: 16 |
| hB7-H5(VISTA) | NM_022153.1 (Gene ID: 64115) | SEQ ID NO: 17 | SEQ ID NO: 18 |
| hKIR: | NM_001322168.1 (Gene ID: 3811) | SEQ ID NO: 19 | SEQ ID NO: 20 |
| hCD137 | NM_001561.5 (Gene ID: 3604) | SEQ ID NO: 21 | SEQ ID NO: 22 |
| hLAG-3 | NM_002286.5 (Gene ID: 3902) | SEQ ID NO: 23 | SEQ ID NO: 24 |
| hTIM-3 | NM_032782.4 (Gene ID: 84868) | SEQ ID NO: 25 | SEQ ID NO: 26 |
| hTIGIT | NM_173799.3 (Gene ID: 201633) | SEQ ID NO: 27 | SEQ ID NO: 28 |
| h0X40 | NM_003327.3 (Gene ID: 7293) | SEQ ID NO: 29 | SEQ ID NO: 30 |
| hBTLA | NM_001085357.1 (Gene ID: 151888) | SEQ ID NO: 31 | SEQ ID NO: 32 |

In the present invention, the “immune checkpoint inhibitor” is not particularly limited, as long as it is a substance that suppresses the expression or activity of the aforementioned immune checkpoint.

The “substance that suppresses the expression of the immune checkpoint” may be one that acts at any level, such as at the level of transcription of the immune checkpoint gene, at the level of posttranscriptional regulation, at the level of translation into protein, and at the level of posttranslational modification. Accordingly, examples of the substance that suppresses the expression of the immune checkpoint include a substance that inhibits the transcription of the gene, a substance that inhibits the processing from the initial transcription product to mRNA, a substance that inhibits the transportation of the mRNA to the cytoplasm, a substance that promotes degradation of the mRNA, a substance that inhibits the translation from the mRNA to the protein, a substance that inhibits the posttranslational modification of the immune checkpoint, and the like. A substance that acts on at any of the levels may be preferably used, but a substance that inhibits the translation from the mRNA to the protein is preferred in terms of direct inhibition of the production of the immune checkpoint.

Examples of the substance that specifically inhibits the translation from the mRNA of the immune checkpoint to the protein include a nucleic acid comprising a nucleotide sequence complementary or substantially complementary to the nucleotide sequence of the mRNA encoding the immune checkpoint or a part thereof.

Examples of the nucleotide sequence complementary or substantially complementary to the nucleotide sequence of the mRNA of the immune checkpoint include

(a) nucleotide sequences complementary or substantially complementary to the nucleotide sequence encoding the immune checkpoint, or
(b) a nucleotide sequence that hybridizes with a complementary strand sequence of the nucleotide sequence encoding the immune checkpoint under highly stringent conditions and that is complementary or substantially complementary to a nucleotide sequence encoding a protein having substantially same activity with the immune checkpoint.

The “substantially complementary” refers to having a complementarity of about 70% or more, preferably about 80% or more, more preferably about 90% or more, most preferably about 95% or more between nucleotide sequences. Moreover, the “activity” refers to the effect of suppressing the anti-tumor activity of T cells to cancer cells. The “substantially same” indicates that the activity thereof is qualitatively (e.g., physiologically or pharmacologically) the same. Therefore, quantitative factors such as the degree of activity (e.g., about 0.1 to about 10 times, preferably about 0.5 to about 2 times) or the molecular weight of the protein may be different. The measurement of the immune checkpoint activity may be conducted according to the methods known per se.

Examples of the highly stringent conditions include hybridization at 45° C. in 6×SSC (sodium chloride/sodium citrate) and then washing at 65° C. in 0.2×SSC/0.1% SDS once or more.

The “part of a nucleotide sequence complementary or substantially complementary to the nucleotide sequence of the mRNA of the immune checkpoint” is not particularly limited, as long as it can specifically bind to mRNA of the immune checkpoint and not particularly limited in length and position, as long as it can inhibit the translation of the protein from the mRNA, but comprises a part that is at least 10 nucleotides or more, preferably about 15 nucleotides or more, and more preferably about 20 nucleotides or more complementary or substantially complementary to the target sequence in terms of the sequence specificity.

Specifically, preferable example of the nucleic acid comprising a nucleotide sequence complementary or substantially complementary to the nucleotide sequence of the mRNA of the immune checkpoint or a part thereof include any of the following (i) to (iii):

(i) an antisense nucleic acid to the mRNA of the immune checkpoint,
(ii) siRNA to the mRNA of the immune checkpoint,
(iii) a nucleic acid that can generate an siRNA to the mRNA of the immune checkpoint.
(i) Antisense Nucleic Acid to mRNA of Immune Checkpoint

The antisense nucleic acid to the mRNA of the immune checkpoint in the present invention (the antisense nucleic acid according to the present invention) is a nucleic acid comprising a nucleotide sequence complementary or substantially complementary to the nucleotide sequence of the mRNA or a part thereof and having a function of suppressing protein synthesis by forming a specific and stable double-strand and binding to the target mRNA and having the function of suppressing the protein synthesis. The antisense nucleic acid may be any of a double-stranded DNA, a single stranded DNA, a double-stranded RNA, a single stranded RNA, a DNA:RNA hybrid.

The target region of the antisense nucleic acid is not particularly limited in length as long as it can inhibit the translation of the immune checkpoint as a result by hybridizing with the antisense nucleic acid and may be the full-length sequence of the mRNA encoding the protein or a partial sequence thereof, and includes a short sequence of about 10 nucleotides to a long sequence of the total sequence of the mRNA or the initial transcription product. An oligonucleotide consisting of about 10 to about 40 nucleotides, and in particular about 15 to about 30 nucleotides is preferred in consideration of the issues such as ease of the synthesis, antigenicity, and intracellular translocation, but it is not limited thereto. Specifically, the sequences such as a 5′ terminal hairpin loop, 6 nucleotides pair repeats at 5′ terminal, a 5′ terminal untranslated region, the translation initiation codon, a protein coding region, the ORF translation stop codon, a 3′ terminal untranslated region, a 3′ terminal palindromic region, or a 3′ terminal hairpin loop of the immune checkpoint can be selected as a preferable target region of the antisense nucleic acid, but it is not particularly limited thereto.

The nucleotide molecule that composes the antisense nucleic acid may be natural DNA or RNA, but may have various kinds of chemical modification to improve stability (chemically and/or anti-enzymatically) and specific activity (affinity to RNA). The antisense nucleic acids may be in a form of antigene. Any of the antisense nucleic acid comprising such modification may be chemically synthesized by a technique known per se.

(ii) siRNA to mRNA of Immune Checkpoint

As used herein, a double-stranded RNA composed of an oligo-RNA complementary to the mRNA of the immune checkpoint and a complementary strand thereof, so-called siRNA, also comprises a nucleotide sequence of the mRNA of the immune checkpoint and a complementary or substantially complementary nucleotide sequence thereof or a part thereof is also defined to be included in the nucleic acid. The siRNA may be designed using commercially available software (e.g.: RNAi Designer; Invitrogen) based on the nucleotide sequence information about the target mRNA.

The ribonucleotide molecule composing the siRNA may also be modified to improve its stability and specific activity similarly to the above-mentioned antisense nucleic acid. However, the RNAi activity may be lost from siRNA in which all ribonucleotide molecules in the natural type RNA are replaced with modified molecules and therefore the introduction of the minimum modified nucleotides that allows the RISC complex to function is necessary.

siRNA can be prepared by respectively synthesizing a sense strand and an antisense strand of the target sequence of the mRNA with a DNA/RNA automatic synthesizer, denaturing at about 90 to about 95° C. for about 1 minute in an appropriate annealing buffer solution, and then annealing at about 30 to about 70° C. for about 1 to about 8 hours. Moreover, the siRNA can also be prepared by synthesizing short hair pin RNA (shRNA) to be a precursor of siRNA and cutting with the dicer.

(iii) Nucleic Acid Capable of Generating siRNA to mRNA of Immune Checkpoint

As used herein, nucleic acids designed such that siRNA to the mRNA of the above-mentioned immune checkpoint is produced in the body is also defined to be included in the nucleic acid comprising a nucleotide sequence complementary or substantially complementary to the nucleotide sequence or a part of the mRNA of the immune checkpoint. Examples of such nucleic acids include expression vectors that have been constructed to express the aforementioned shRNA, and the like. shRNA can be prepared by designing an oligo-RNA comprising a nucleotide sequence in which a sense strand and an antisense strand of the spacer sequence of the target sequence on the mRNA are connected by inserting a spacer sequence capable of forming an appropriate loop structure (e.g., about 15 to 25 nucleotides) and synthesizing this with an automatic DNA/RNA synthesizer. The expression vector comprising an expression cassette of shRNA can be prepared by producing a double-stranded DNA encoding the aforementioned shRNA by a conventional method and inserting it in an appropriate expression vector. As an expression vector of the shRNA, a vector having a Pol III promoter such as U6 or H1 origin can be used.

The inhibitory activity of these nucleic acids on the expression of the immune checkpoint can be examined by using a transformant in which a gene of the immune checkpoint has been introduced, in vivo and in vitro gene expression systems of the immune checkpoint, or an in vivo or in vitro protein translation system of the immune checkpoint.

The substance that suppresses the expression of the immune checkpoint according to the present invention is not limited to the aforementioned nucleic acids and may be another substance such as a low molecular weight compound as long as it directly or indirectly inhibits the production of the immune checkpoint.

In the present invention, the “substance that suppresses activity of the immune checkpoint” may be any substance as long as it suppresses an effect of controlling the anti-tumor activity for the cancer cell of the T cell.

Specifically, the “substance that suppresses the activity of the immune checkpoint” includes an antibody specific to the immune checkpoint. The antibody may be either a polyclonal antibody or a monoclonal antibody. These antibodies can be produced according to the manufacturing process of antibodies or antisera publicly known per se. The isotype of the antibody is not particularly limited, but examples thereof include preferably IgG, IgM, or IgA, and particularly preferably IgG. Moreover, the antibody is not particularly limited as long as it has at least a complementarity-determining region (CDR) that specifically recognizes and bind to a target antigen, and may be a complete antibody molecule as well as a fragment such as Fab, Fab′, or F(ab′)₂, a conjugate molecule produced by genetic engineering, such as scFv, scFv-Fc, a minibody, or a diabody, or a derivative thereof that is modified with a molecule having the protein stabilization effect such as polyethyleneglycol (PEG).

Preferred examples of the “immune checkpoint inhibitor” include an anti-PD-1 antibody, an anti-PD-L1 antibody, an anti-PD-L2 antibody, an anti-CTLA-4 antibody, an anti-KIR antibody, an anti-CD137 antibody, an anti-LAG-3 antibody, an anti-OX40 antibody, an anti-CD80 antibody, an anti-CD86 antibody, an anti-B7-H3 antibody, an anti-B7-H4 antibody, an anti-B7-H5 antibody, an anti-TIM-3 antibody, an anti-TIGIT antibody, and an anti-BTLA antibody. Among them, an anti-PD-1 antibody, an anti-PD-L1 antibody, and an anti-CTLA-4 antibody are preferred and an anti-PD-1 antibody is more preferred.

The “anti-PD-1 antibody” used in the present invention is not particularly limited as long as it can bind to PD-1 and inhibit the function as an immune checkpoint. Examples of the anti-PD-1 antibody include those already approved and sold as a medicament and those under development and they can be used suitably. Examples of such an “anti-PD-1 antibody” include, but are not limited to, Nivolumab (Opdivo (GSK/Ono pharmaceutical)), Pembrolizumab (MK-3475 (Merck)), which is a humanized IgG4 antibody, Pidilizumab (CT-011 (CureTech)), REGN-2810/SAR-439684 (Regeneron), PDR-001 (Novartis), AMP-514/MEDI-0680 (Amplimmune), TSR-042 (AnaptysBio), PF-06801591 (Pfizer), JS-001 (Shanghai Junshi Biosciences), IBI-308 (Innovent Biologics), and BGB-A317 (BeiGene).

The “anti-PD-L1 antibody” to be used in the present invention is not particularly limited, as long as it can bind to PD-L1 and inhibit the function as an immune checkpoint. Example of the anti-PD-L1 antibody include those already approved (sold) as a medicament and those under development and they can be used suitably. Examples of such an “anti-PD-L1 antibody” include, but are not limited to, atezolizumab (MPDL3280A/RG-7446 (Roche/Chugai Pharmaceutical Co., Ltd.), Durvalumab (MEDI4736 (AstraZeneca)), avelumab (MSB0010718C (Merck)), MED10680/AMP-514 (Medimmune), MDX-1105/BMS-936559 (BMS), INCAGN-1876 (Agenus), LY-3300054 (Eli Lilly), and CA-170 (Aurigene Discovery Technology)).

The “anti-CTLA-4 antibody” to be used in the present invention is not particularly limited as long as it can bind to CTLA-4 and inhibit the function as an immune checkpoint. Example of the anti-CTLA-4 antibody include those already approved (sold) as a medicament and those under development and they can be used suitably. Examples of such an “anti-CTLA-4 antibody” include, but are not limited to, Ipilimumab (MDX-010), tremelimumab (CP675/206 (Pfizer)), and AGEN-1884 (4-Antibody).

Besides those described above, Lirilumab (IPH2102/BMS-986015), which is an anti-killer cell immunoglobulin-like receptor (KIR) antibody, Urelumab (BMS-663513) and PF-05082566, which are anti-CD137 antibodies, BMS-986016, which is an anti-LAG-3 antibody, MEDI6469, which is an anti-OX40 antibody, MSB0011359C/M-7824(Merck), which is a bi-functional fusion protein that targets PD-LA and TGF-β are under development as an immune checkpoint inhibitor.

The antibodies described above may be produced by a conventional method. In that case, the antibody is preferably a chimeric antibody, a humanized antibody, or a fully human antibody to reduce the xenogeneic antigenicity to humans.

The “immune checkpoint inhibitor” according to the present invention may comprise a pharmacologically acceptable carrier and/or an additive. Examples of such a carrier and an additive include a filler, a binder, a lubricant, a solvent, a disintegrator, a solubilizing agent, a suspending agent, an emulsifier, an isotonizing agent, a stabilizer, a soothing agent, an antiseptic, antioxidant, a corrigent, a colorant, a buffer, a superplasticizer, and the like, but are not limited thereto, and other commonly used carriers and/or additives may be used. The specific examples of the carrier and additive are as described above.

The route of administration of the “immune checkpoint inhibitor” of the present invention is not particularly limited. Parenteral administration is preferred. Specific examples of the parenteral administration include injection, transnasal administration, pulmonary administration, transdermal administration, and the like. Examples of the injection include intravenous injection, intramuscular injection, intraperitoneal injection, and subcutaneous injection. The mode of administration can be selected according to the age and symptoms of the patient.

When an immune checkpoint inhibitor and HVJ-E are contained and provided in separate agents in the medicament according to the present invention, the dosage forms and the routes of administration of these agents may be different or the same. Moreover, one or more different preparations may further be combined.

The dose of the “immune checkpoint inhibitor” according to the present invention can be determined based on the purpose of use and the route of administration thereof. The dose is adjusted to provide an optimal response (for example, therapeutic response) desired. Since the active ingredient is an antibody, the dose is in the range of about 0.0001 to 100 mg/kg and usually 0.01 to 5 mg/kg patient body weight. For example, dose is about 0.3 mg/kg of body weight, 1 mg/kg of body weight, 3 mg/kg of body weight, 5 mg/kg of body weight or 10 mg/kg of body weight, or in the range of 1 to 10 mg/kg. Typical regimens are, for example, once a week administration, once in 2 weeks administration, once in 3 weeks administration, once in 4 weeks administration, once a month administration, once in 3 months administration, or once in 3 to 6 months administration. For example, in the case of administration by IV infusion, the dose is 1 mg/kg body weight or 3 mg/kg body weight. The dose frequency can be determined depending on the symptoms. The inhibitor may be administered in a single bolus or divided into several doses to take time. For example, it may be administered 6 times every 4 weeks and then administered every 3 weeks, or may be administered every 3 weeks, or may be administered once at 3 mg/kg body weight and then 1 mg/kg of body weight every 3 weeks.

When HVJ-E and the immune checkpoint inhibitor according to the present invention are administered in combination, the order of administration is not limited, and HVJ-E may be administered after the administration of the immune checkpoint inhibitor, or both may be administered simultaneously, or the immune checkpoint inhibitor may be administered after the administration of HVJ-E. Preferably, the immune checkpoint inhibitor is administered after the administration of HVJ-E.

When HVJ-E and the immune checkpoint inhibitor are sequentially administered, the interval of the administration of HVJ-E and the immune checkpoint inhibitor is not particularly limited and may be set so that desired combinational effect is obtained, in consideration of the route of administration, the dosage form, the dose, the residual concentration, and the like. For example, the interval of administration is 0 hours to 7 days, preferably 0 hours to 3 days, more preferably 0 to 24 hours, and further preferably 0 hours to 12 hours.

The interval of administration may be determined, for example, based on the result of the analysis of the blood concentration of the agent or a metabolite thereof in the patient by a known method.

### EXAMPLES

Hereinafter, the present invention will be described in detail with reference to Examples, but the present invention is not limited by these Examples.

### Example 1

Improving effect of administration of single agent of either HVJ-E or anti-PD-1 antibody and combined administration thereof on learning disability in β amyloid single administered model mice 1. Material and method Test substance:

(1) HVJ-E (batch number: 140523, Genomldea, Inc.)
(2) LEAF™ Purified anti-mouse CD279 (anti-PD-1 antibody, batch number: B203991, BioLegend Inc.) Positive control substance

5 mg Aricept® tablet (donepezil hydrochloride, batch number: 61A53K, Eisai Co., Ltd.) Vehicle:

(1) Metolose® SM-100 (methylcellulose (hereinafter referred to as MC), batch number 5065340, Shin-Etsu Chemical Co., Ltd.)
(2) Water for injection (batch number: 5C74N, Otsuka Pharmaceutical Factory, Inc.)
5% trehalose solution (batch number: 160523, Genomldea, Inc.)
(3) Phosphate-buffered salt (batch number: 1259290, DS Pharma Biomedical Co., Ltd.) Substance for model preparation:

Amyloid-β Protein (25-35) (β amyloid, batch number: AW14089, PolyPeptide Laboratories) Vehicle for substance for model preparation:

Water for injection (batch number: K5F71, Otsuka Pharmaceutical Factory, Inc.)

### Administration Sample:

### Preparation of Vehicle (0.5% MC)

A required amount of MC is weighed (electronic balance: either XP205DR or PB3002-S/FACT is used, Mettler Toledo International Inc.) and then stirred and prepared to be at a concentration of 0.5 w/v % with water for injection. The prepared 0.5% MC solution is kept under refrigeration (control temperature: 2.0 to 8.0° C.) and used within 7 days after the preparation.

### Preparation of Vehicle (PBS)

Tablets of phosphate-buffered salt are dissolved with 100 mL per tablet of water for injection and sterilized by filtration with a filter (MILLIPORE) with a pore size of 0.22 μm.

### Preparation of Test Substance (HVJ-E)

A required number of test substances are brought to room temperature and aluminum caps are completely removed from the vials leaving only rubber stoppers. A 1 mL disposable syringe (Terumo Corporation) made of polypropylene with a needle (Terumo Corporation) is filled with 1 mL of water for injection. The outside of the rubber stopper is cleaned with ethanol cotton, then the rubber stopper is penetrated with the aforementioned needle attached to the 1 mL disposable syringe made of polypropylene, and the 1 mL per vial of water for injection is added such as to go down along the wall. The vial is swirled in circle while avoiding making bubbles in the liquid and attaching the liquid to the rubber stopper to completely dissolve the substance and yield a homogeneous white solution. The concentration of HVJ-E in this preparation is 10,000 mNAU/mL (containing 5% trehalose). The rubber stopper is removed and the whole content is transferred into a tube made of polypropylene using a micropipet while avoiding making bubbles in the liquid.

The tube in which the solution has been transferred is immediately placed on ice and diluted with a 5% trehalose aqueous solution to prepare a dosing solution at a predetermined concentration (2,000 mNAU/mL). It is stored on ice after the preparation and brings back to room temperature just before the administration. Preparation of test substance (anti-PD-1 antibody)

The antibody (1 vial: 1 mg/mL) is used as it is. It is stored on ice till the administration and brings back to room temperature just before the administration.

### Preparation of Positive Control Substance

It is prepared in terms of a salt thereof (conversion factor: 1.10).

The required number of donepezil tablets is placed in an agate mortar and sufficiently ground and 0.5% MC is gradually added to a predetermined concentration.

### Preparation of Substance for Generation of Model

β amyloid is dissolved to 2 mM with water for injection and the mixture is incubated at 37° C. for 4 days to prepare a β amyloid solution. The operation is conducted in a clean bench and instruments and containers to be used are those that have been sterilized.

### Frequency of Preparation

0.5% MC is prepared once or more every week, the substance for the generation of the model is prepared once 4 days before the administration, and the vehicle (PBS), the test substance, and the positive control substance are prepared every time when needed.

### Test System

Animal species and strain

### Mouse (SPF): Slc: ddY

Male, 5 weeks of age, 50 animals
Range of body weights one day after the acquisition (23 to 28 g)

### Source: Japan SLC, Inc.

### Quarantine Inspection and Acclimation

The animals acquired are put in quarantine for a period of 5 days and acclimated during the following 4 days. During this period, the body weight is measured (an electronic balance: one of PB3002, PG2002-S, PB3002-S/FACT, and MS16025/02 is used, Mettler Toledo International Inc.) 3 times and general conditions are examined once a day as quarantine inspection and acclimation. Animals confirmed to have no change in body weight and no abnormality in general conditions are assigned to groups. Animals found to have change in body weight and abnormality in general conditions are euthanized using gaseous carbonic acid.

### Group Assignment

The group assignment is conducted after stratification based on the body weight using a computing system (IBUKI, Nihon Bioresearch Inc.) such that the average body weights of the groups are almost identical by random sampling. Excess animals after the group assignment are euthanized using gaseous carbonic acid on the day of group assignment.

### Identification of Individual Animals

Animals are identified by a combination of marking on the tail with an oil-based ink and application of a dye on a limb on the day of acquisition. After the group assignment, animals are identified by labeling of their tails with the animal numbers with an oil-based ink in the same color as their cage labels (except the sham operation group, which is labeled with a black ink). The cages are labeled with labels on which the test numbers, the acquisition date and the animal numbers for quarantine inspection and acclimation are written during the quarantine inspection and acclimation period and labeled with labels in different colors between the groups on which the test numbers, the group names, the doses, and the animal numbers are written after the group assignment.

### Environmental Conditions and Maintenance of Animals

Animals are maintained in a breeding room (Room 9 in Building E except during the quarantine inspection period, in which animals are maintained in Room 10 in Building E) maintained under the following conditions: controlled temperature: 20.0 to 26.0° C., controlled humidity: 40.0 to 70.0%, light and dark for 12 hours each (lighting: 6:00 a.m. to 6:00 p.m.), and the number of ventilation: 12 times/h (with fresh air through a filter). Up to 10 animals per cage or up to 5 animals per cage after the group assignment are maintained in groups in plastic cages (W: 310×D: 360×H: 175 mm) in which an autoclaved floor sheet is placed. The feeders are changed once or more in 2 weeks and the water supply bottles and the plastic cages are changed twice or more per week. Cleaning and sterilization of the animal breeding room are conducted every day.

### Feeding-Stuff

Solid feed (MF, Oriental Yeast Co., Ltd.) containing no milk protein within 5 months after the production was placed in feeders and animals are fed freely. It is confirmed that the concentrations of pollutants, the number of bacteria, and the content of nutritional ingredients in feeding-stuff meet the standards of the test facilities for all lots of the feeding-stuff used.

Analysis organization: Eurofins Scientific Analytics (pollutants) and Oriental Yeast Co., Ltd. (the number of bacteria and nutrition ingredients)

### Route of Administration

Vehicle (0.5% MC) and positive control substance: oral

Forced oral administration is conducted using a disposable syringe (Terumo Corporation) made of polypropylene with a disposable oral probe (Fuchigami Kikai Co., Ltd.) for mice. At the time of the administration operation, each administration sample is mixed by inversion for each animal and aspirated in a syringe.

Dosing amount of liquid: calculated as 10 mL/kg from the body weight on the day of administration
Dose frequency: total 11 times of once a day
Time of administration: between 9:00 a.m. and 12:00 a.m., but administration on the day of β amyloid injection and the day of examination is as follows.

On the day of β amyloid injection, administration is conducted after the β amyloid injection (after emergence).

On the day of examination, administration is conducted about 1 hour (+10 minutes) before the measurement.

Reason of selection: it is a usual method used in the test facilities.
Vehicle (5% trehalose solution) and HVJ-E: Subcutaneous (dorsocervical area)

Subcutaneous administration in the dorsocervical area is conducted using a disposable syringe (Terumo Corporation) made of polypropylene with a disposable needle (Terumo Corporation). At the time of the administration operation, each administration sample is mixed by inversion for each animal and aspirated in a syringe.

Dosing amount of liquid: calculated as 5 mL/kg from the body weight on the day of administration
Dose frequency: total 6 times of once in 2 days.
Time of administration: between 9:00 a.m. and 12:00 a.m., but administration on the day of β amyloid injection and the day of examination is as follows.

On the day of β amyloid injection, administration is conducted after the β amyloid injection (after emergence).

On the day of examination, administration is conducted about 1 hour (+10 minutes) before the measurement.

### Vehicle (PBS) and Anti-PD-1 Antibody: Intraperitoneal

Intraperitoneal administration is conducted using a disposable syringe (Terumo Corporation) made of polypropylene with a disposable needle (Terumo Corporation). At the time of the administration operation, each administration sample is mixed by inversion for each animal and aspirated in a syringe.

Dosing amount of liquid: 0.25 mL per animal.
Dose frequency: Total twice of once on Day 1 of administration and once on Day 4 of administration.
Time of administration: between 9:00 a.m. and 12:00 a.m.
Reason of selection: It is a usual method used in the test facilities.

TABLE 2
Group configuration and dose

| | | | | Number |
|---|---|---|---|---|
| | | | Route of | of |
| | Name of group | Dose | administration | animals |
| 1. | Vehicle control* | — | Oral | 8 |
| 2. | HVJ-E** | 10,000 mNAU/kg | Subcutaneous + | 8 |
| | | | intraperitoneal | |
| 3. | Anti-PD-1 | 250 μg/body | Intraperitoneal + | 8 |
| | antibody*** | | subcutaneous | |
| 4. | HVJ-E + | 10,000 mNAU/kg + | Subcutaneous + | 8 |
| | Anti-PD-1 | 250 μg/body | intraperitoneal | |
| | antibody | | | |
| 5. | Donepezil | 0.5 mg/kg | Oral | 8 |

*0.5% MC is orally administered.
**HVJ-E is subcutaneously administered and the vehicle (PBS) is intraperitoneally administered.
***The anti-PD-1 antibody is intraperitoneally administered and the vehicle (5% trehalose solution) is subcutaneously administered in the dorsocervical area.

### Reason for Dose Setting

The doses of HVJ-E and the anti-PD-1 antibody were set at doses that were expected to provide sufficient pharmacological effects. The dose of donepezil was set based on the background data at the test facilities at a dose that was expected to provide a sufficient pharmacological effect.

### Items of Observation and Examination

### Experimental Schedule

The starting day of the administration of the administration sample is referred to as Day 1 of administration and β amyloid is injected on Day 3 of administration. Subsequently, the Y-maze test is conducted on Day 9 of administration, the passive avoidance test is conducted on Day 10 to 11 of administration, the brain is extirpated after the passive avoidance test (retention trial).

### General Conditions

General conditions are observed before the administration once a day.

When a mouse is dying (a state in which the self-motion is decreased, the breathing and/or the pulse are abnormal, the body temperature is lowered, the animal is in a side-lying position, a prone position, or the like, and the response to external stimulation is decreased), the state is determined as a humanitarian endpoint and the animal is euthanized using gaseous carbonic acid.

### Measurement of Body Weight

The body weight is measured (an electronic balance: one of PB3002, PG2002-S, PB3002-S/FACT, and MS16025/02 is used, Mettler Toledo International Inc.) every day of administration. The body weight is measured before the administration.

### Injection of β Amyloid

40 mg/kg of pentobarbital sodium (Tokyo Chemical Industry Co., Ltd.), is intraperitoneally administered (dosing amount of liquid: 10 mL/kg) to animals to anesthetize the animals. After anesthesia, levobupivacaine hydrochloride (Popscaine®, 0.25% injection, Maruishi Pharmaceutical Co., Ltd.) is subcutaneously administered (0.1 mL) to the scalp. Parietal hairs of the animal are removed and the head is fixed with a brain stereotaxis apparatus. The scalp is cut open after sterilization with a tincture of iodine to expose the skull and the connective tissue on the skull is removed with a cotton swab. The skull is then dried with a blower to find the position of bregma more easily. Using dental drill, a hole for inserting a stainless steel pipe is made on the skull at the position 1 mm side (the right side) and 0.2 mm posterior to bregma. The stainless steel pipe connected to a silicon tube having an outside diameter of 0.5 mm and a microsyringe is perpendicularly inserted to a depth of 2.5 mm from the bone surface. 3 μL of a β amyloid solution (6 μmol/3 μL) is injected into the cerebral ventricle with a microsyringe pump for 3 minutes. After injection, the stainless steel pipe is left inserted for 3 minutes and then taken out slowly. Subsequently, the stainless steel pipe is removed, the cranial hole is plugged with a non-absorbable bone marrow styptic (Nestop®, Alfresa Pharma Corporation), and the scalp is sutured. The animal is removed from the brain stereotaxis apparatus and returned to the animal cage. The stainless steel pipe and the silicon tube are used after sterilization.

### Y-Maze Test (Spontaneous Alternation Test)

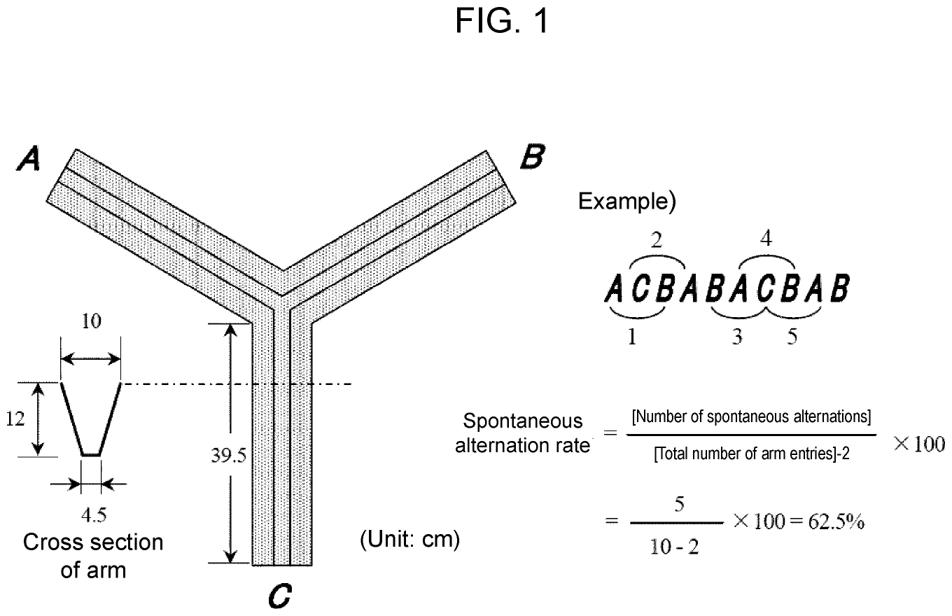

### Apparatus (FIG. 1).

For the test, a plastic Y-shaped maze (UNICOM) having 3 arms each diverging at an angle of 120° and having a length of 39.5 cm, in which the width of the floor is 4.5 cm and the height of the wall is 12 cm, is used. Before the measurement, the illuminance of the floor of the apparatus is adjusted to 10 to 40 Lux.

### Method of Measurement

The test is performed about 1 hour after the administration. An animal is placed in one of the arms of the Y-shaped maze and allowed to explore in the maze freely for 8 minutes. The order of the arms in which the animal moves within the measurement time is recorded and the number of times of moving to another arm is counted as the total number of entries. Next, combinations of 3 different arms consecutively selected by the animal are examined in this record and the number of the combinations is expressed as the spontaneous alternation. Furthermore, the spontaneous alternation rate is calculated using the following equation.

Spontaneous alternation rate (%)=[the number of spontaneous alternation/(the total number of entries−2)]×100

### Passive Avoidance Test

For the test, a step through passive avoidance response apparatus (with dark and light compartments: a product made in company, SHOCK SCRAMBLER: UNICOM) having a light compartment (W: 100×D: 100×H: 300 mm) and a dark compartment (W: 240×D: 245×H: 300 mm) which provide electrical stimulation from the grid of the floor, partitioned by a central Guillotine door is used. An animal is placed in the light compartment and the Guillotine door is calmly opened 10 seconds later. The time before the animal enters the dark compartment (response latency) is measured.

In the acquisition trial (Day 1), the Guillotine door is closed at the same time as the animal enters the dark compartment and electrical stimulation (0.2 mA, 2 sec, a scramble method) is provided. The presence or absence of cry of the animal upon the electrical stimulation is recorded. The response latency is up to 300 seconds.

The retention trial (Day 2) is terminated when the animal enters the dark compartment or retained in the light compartment for 300 seconds.

Animals that finished the retention trial are euthanized using gaseous carbonic acid.

### Analysis of Result

For each evaluation category, the average and the standard error of each group are calculated and analyzed as follows.

### Statistical Analysis

The significance test is conducted by comparison tests between 2 groups on the vehicle control group and the HVJ-E group, the vehicle control group and the anti-PD-1 antibody group, the vehicle control group and the HVJ-E+ anti-PD-1 antibody group, the HVJ-E group and the HVJ-E+ anti-PD-1 antibody group, the anti-PD-1 antibody group and the HVJ-E+ anti-PD-1 antibody group, and the vehicle control group and the donepezil group.

For the comparison tests between 2 groups, the homoscedasticity is tested by the F test and Student's t-test is conducted when homoscedastic and Aspin-Welch test is conducted when heteroscedastic.

The level of significance is set at 5% and the results are described as less than 5% (p<0.05) and less than 1% (p<0.01). The significance test is conducted using a commercially available statistics program (SAS system, SAS Institute Japan Ltd.).

Results

### (1) Y-Maze Test (the Number of Spontaneous Alternation, Spontaneous Alternation Rate)

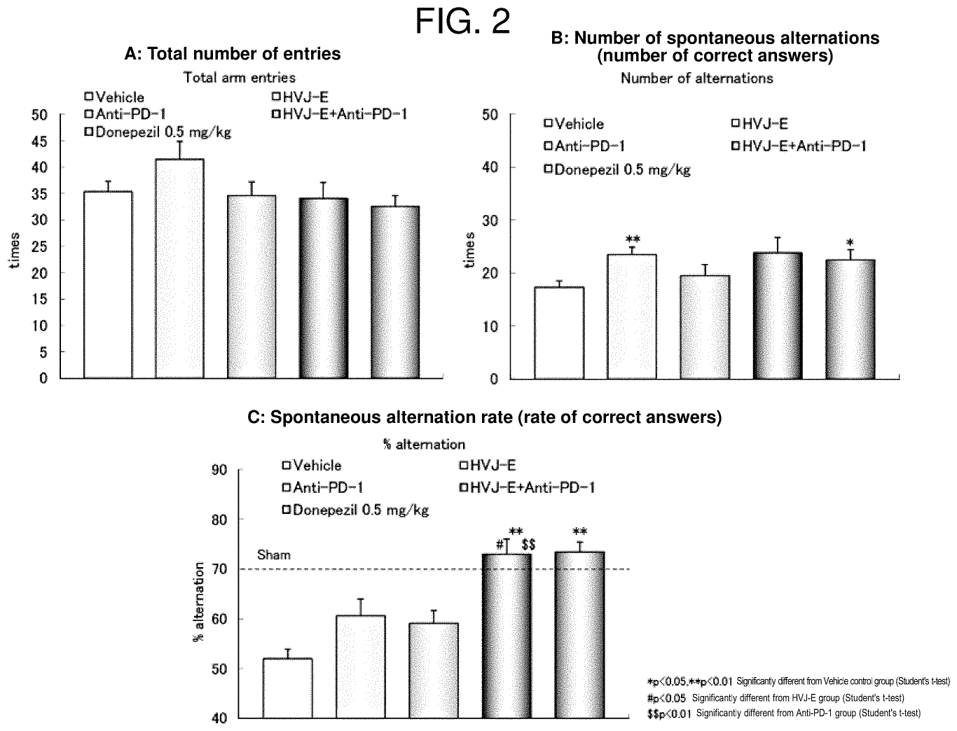

The result of the Y-maze test is shown in FIG. 2. The HVJ-E+ anti-PD-1 antibody combined application group exhibited a significantly higher spontaneous alteration rate (P<0.01) in comparison with the vehicle control group and the numerical value was equivalent to the donepezil group. Moreover, the spontaneous alternation rate of the HVJ-E+ anti-PD-1 antibody combined application group exhibited a significantly higher spontaneous alternation rate in comparison with the single administration (HVJ-E group or anti-PD-1 antibody group) and the positive interaction was found in Additive interaction and Multiplicative interaction, indicating synergistic effect.

### (2) Passive Avoidance Test (Response Latency)

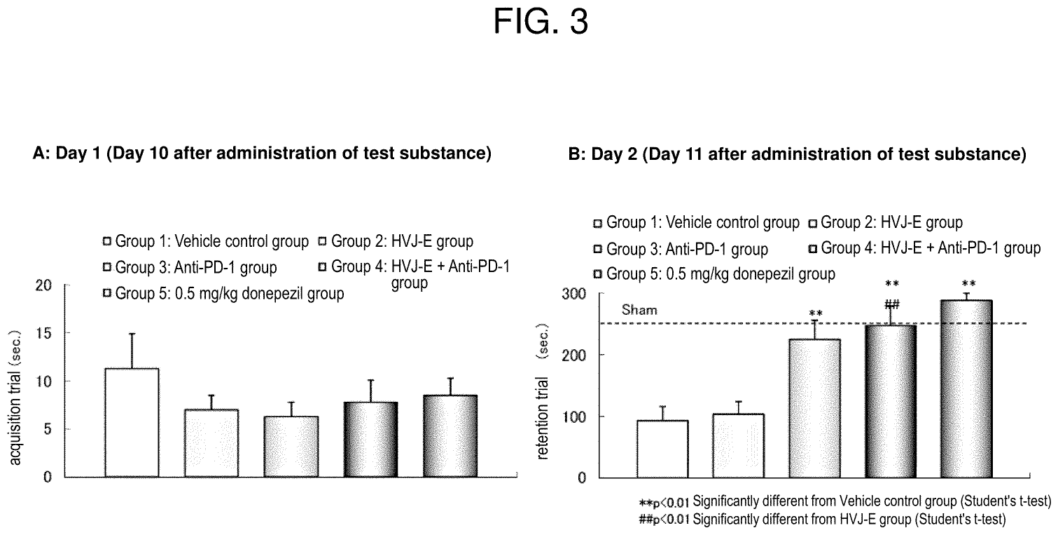

The result of the passive test (response latency) is shown in FIG. 3. The anti-PD-1 antibody group and the HVJ-E+ anti-PD-1 antibody combined application group exhibited a significantly higher effect in comparison with the vehicle control group, although they were slightly inferior to the donepezil group. Moreover, the response latency of the HVJ-E+ anti-PD-1 antibody combined application group was significantly different from that of the HVJ-E single administration group.

### DISCUSSION

Schenk et al. have reported that immunizing Alzheimer model mice made by overproduction of AP with Aβ₁₋₄₂ resulted in pathological improvement (Nature 400: 173-, 1999, listed above) and behavioral improvement (Nature 408:979- and 982-, 2000). Furthermore, they have reported that peripheral administration of an anti-Aβ antibody resulted in pathological improvement (Nature Med 6: 916-, 2000). Moreover, Baruch et al. have reported that administration of a PD-1 antibody to Alzheimer model mice resulted in pathological improvement (Nature Med 22: 135-, 2016, listed above).

Based on the results of this Example and the fact described above, it is considered that both of HVJ-E and the anti-PD-1 antibody (immune checkpoint inhibitor) remove Aβ, which is a foreign substance by activating natural immunity as well as acquired immunity, and improve pathological conditions of Alzheimer disease. However, the combined application of HVJ-E and the anti-PD-1 antibody had a significantly higher effect in comparison with single administration, and the fact suggests the possibility that mechanisms of action of both are different. Moreover, HVJ-E exhibited highly improving effect even in single administration, and the fact suggests that HVJ-E contributes to improvement of pathological conditions by a pathway other than the activation of acquired immunity alone. By this experiment, it was shown that more effective treatment of cognitive impairment and neurodegenerative diseases with accumulation of a prionoid such as Aβ, including Alzheimer-type dementia, may be possible by use of HVJ-E alone or in combination with an immune checkpoint inhibitor.

### Example 2

Improving effect of single and combined administration of HVJ-E and anti-PD-1 antibody on learning disability in β amyloid single administration model mice (comparison between HVJ-E subcutaneous administration and intranasal administration)

The Y-maze test was conducted in the same method described in Example 1, except that a HVJ-E intranasal administration group was created in addition to the HVJ-E subcutaneous administration group. The route of administration and group configuration are shown below.

### Route of Administration

### Vehicle (0.5% MC) and Positive Control Substance: Oral

The same procedure described in Example 1

### Vehicle (5% Trehalose Solution) and HVJ-E: Intranasal or Subcutaneous

The intranasal administration is conducted by administration into a nasal cavity using a micropipet (Eppendorf AG). Subcutaneous administration is conducted using a disposable syringe (Terumo Corporation) made of polypropylene with a disposable needle (Terumo Corporation). At the time of the administration operation, each administration sample is mixed for each animal and aspirated in a syringe.

The intranasal administration is conducted under the following conditions.

Dosing amount of liquid: administration is conducted into both of the nasal cavities (10 μL/nose) with a liquid amount of 5 μL per cavity of an animal.
Dose frequency: total 9 times of once a day
The subcutaneous administration is conducted under the following conditions.
Dosing amount of liquid: calculated as 5 mL/kg from the body weight on the day of administration
Dose frequency: total 6 times of once in 2 days.

The time of either of the intranasal administration and the subcutaneous administration is between 9:00 a.m. and 12:00 a.m., but administration on the day of 0 amyloid injection and on the day of examination is conducted as follows.

On the day of β amyloid injection, administration is conducted after the β amyloid injection (after emergence).

On the day of examination, administration is conducted about 1 hour (+10 minutes) before the measurement.

### Vehicle (PBS) and Anti-PD-1 Antibody: Intraperitoneal

The same procedure described in Example 1

TABLE 3
Group configuration and dose

| | | | | Number |
|---|---|---|---|---|
| | | | Route of | of |
| | Name of group | Dose | administration | animals |
| 1. | Sham-operation | — | Intranasal + | 8 |
| | | | intraperitoneal | |
| 2. | Vehicle control | — | Intranasal + | 8 |
| | | | intraperitoneal | |
| 3. | HVJ-E*** | 100 mNAU/body | Intranasal + | 8 |
| | | | intraperitoneal | |
| 4. | HVJ-E**** + | 100 mNAU/body + | Intranasal + | 8 |
| | Anti-PD-1 antibody | 250 μg/body | intraperitoneal | |
| 5. | Donepezil | 0.5 mg/kg | Oral | 8 |
| 6. | HVJ-E***** + | 10,000 mNAU/kg + | Subcutaneous + | 8 |
| | Anti-PD-1 antibody | 250 μg/body | intraperitoneal | |

*The vehicle (5% trehalose solution) is intranasally administered and the vehicle (PBS) is intraperitoneally administered.
**0.5% MC is orally administered.
***HVJ-E is intranasally administered and the vehicle (PBS) is intraperitoneally administered.
****HVJ-E is intranasally administered and the anti-PD-1 antibody is intraperitoneally administered.
*****HVJ-E is subcutaneously administered and the anti-PD-1 antibody is intraperitoneally administered.

### Results

### Y-Maze Test (the Number of Spontaneous Alternation, Spontaneous Alternation Rate)

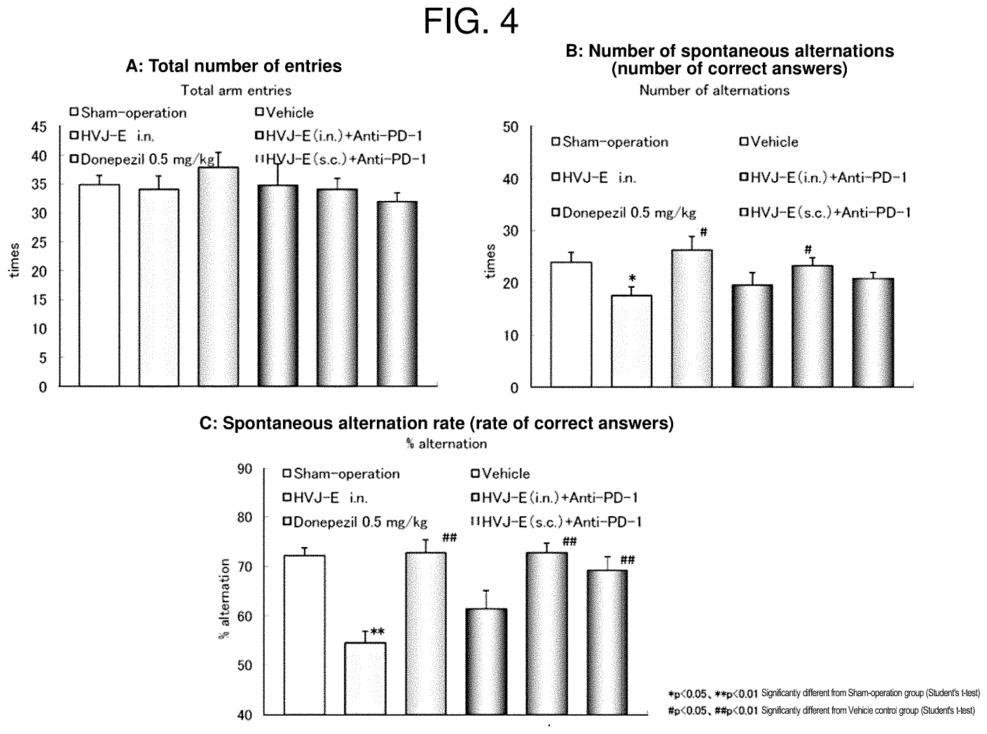

The result of the Y-maze test is shown in FIG. 4. The HVJ-E intranasal administration group exhibited significantly higher number of spontaneous alternation (P<0.05) and spontaneous alternation rate (P<0.01) in comparison with the vehicle control group and the numerical value was equivalent to the donepezil group. Meanwhile, in the combined application with the anti-PD-1 antibody, the HVJ-E (intranasal administration)+ anti-PD-1 antibody administration group was not significantly different from the vehicle control group, but the HVJ-E (subcutaneous administration)+ anti-PD-1 antibody administration group had a significantly higher spontaneous alternation rate (P<0.01) in comparison with the vehicle control group.

### DISCUSSION

In the experiments described in this Example, it was confirmed that learning disability of mice was markedly improved in the HVJ-E intranasal administration group (the group to which the anti-PD-1 antibody was not administered) in comparison with the vehicle administration group. Moreover, in the experiments described in this Example, it was confirmed that the effect of combined application of HVJ-E and the anti-PD-1 antibody was different between intranasal administration and subcutaneous administration. HVJ-E is considered to attract NK cells, CD4+ T cells, and CD8+ T cells to the tumor lesion by induction of chemokine production from tumor cells, dendritic cells, and macrophages and induce the Th1 dominant transformation to increase antitumor effect of NK cells and Effector T cells (Cancer Res. 2007). Moreover, it has been also reported that HVJ-E acts also on neutrophiles and induces the N1 dominant transformation to increase the antitumor effect (Chang C Y, et al., Oncotarget. 2016 Jul. 5; 7 (27): 42195-42207). PD-1 is considered to be expressed mainly in lymphocytes such as T cells and B cells as well as bone marrow cells and macrophages (Yuan B, et al., Immunol Lett. 2016 November; 179: 114-121), and HVJ-E is also considered to act on various kinds of immune cells as described above. It is considered that mucosa immunity and systemic immunity are simultaneously activated by intranasal administration of HVJ-E and HVJ-liposome (Yasuoka E, et al., J Mol Med (Berl). 2007 March; 85 (3): 283-92, Sakaue G, et al., J Immunol. 2003 Jan. 1, 170 (1) 495-502). It seems that the difference between intranasal administration and subcutaneous administration is related to this mechanism of action of HVJ-E. In either case, HVJ-E is expected to be capable of more effectively treating cognitive impairment and neurodegenerative diseases with accumulation of a prionoid such as Aβ, including Alzheimer-type dementia by optimizing the formulation and the route of administration.

### Example 3

Improving effect of single intranasal administration of HVJ-E on learning disability in the 0 amyloid single administration model mice (dose-response test)

In Examples 1 and 2, single and combined administration of HVJ-E and an anti-PD-1 antibody to animals in which learning disability has been induced by β amyloid single intraventricular administration exhibited more improving effect in the Y-maze test (short-term memory impairment) than the HVJ-E single intranasal administration. In this example, to confirm the dose response of HVJ-E single intranasal administration, the improvement effect of HVJ-E single intranasal administration at various concentrations on the learning disability was evaluated in the Y-maze test using the β amyloid single intraventricular administration mice, which is an Alzheimer disease model.

The Y-maze test was conducted in the same method described in Example 1 except that the dose of HVJ-E is changed. The route of administration and group configuration are shown below.

### Route of Administration

### Positive Control Substance: Oral

The same procedure described in Example 1 Vehicle (5% trehalose solution) and HVJ-E: intranasal

The intranasal administration is conducted by administration into a nasal cavity using a micropipet (Eppendorf AG). Subcutaneous administration is conducted using a disposable syringe (Terumo Corporation) made of polypropylene with a disposable needle (Terumo Corporation) as in Example 1. At the time of the administration operation, each administration sample is mixed for each animal and aspirated in a syringe.

The intranasal administration is conducted under the following conditions.

Dosing amount of liquid: administration is conducted into both of the nasal cavities (10 μL/nose) with a liquid amount of 5 μL per cavity of an animal.
Dose frequency: total 9 times of once a day
Time of administration is between 9:00 a.m. and 12:00 a.m., but administration on the day of β amyloid injection and on the day of examination is conducted as follows.

On the day of β amyloid injection, administration is conducted after the β amyloid injection (after emergence).

On the day of examination, administration is conducted about 1 hour (+10 minutes) before the measurement.

TABLE 4
Group configuration and dose

| | | | Route of | Number of |
|---|---|---|---|---|
| | Name of group | Dose | administration | animals |
| 1. | Vehicle control* | — | Intranasal | 8 |
| 2. | HVJ-E | 10 mNAU/body | Intranasal | 8 |
| 3. | HVJ-E | 50 mNAU/body | Intranasal | 8 |
| 4. | HVJ-E | 100 mNAU/body | Intranasal | 8 |
| 5. | Donepezil | 0.5 mg/kg | Oral | 8 |

*The vehicle (5% trehalose solution) is intranasally administered.

### Results

### Y-Maze Test (the Number of Spontaneous Alternation, Spontaneous Alternation Rate)

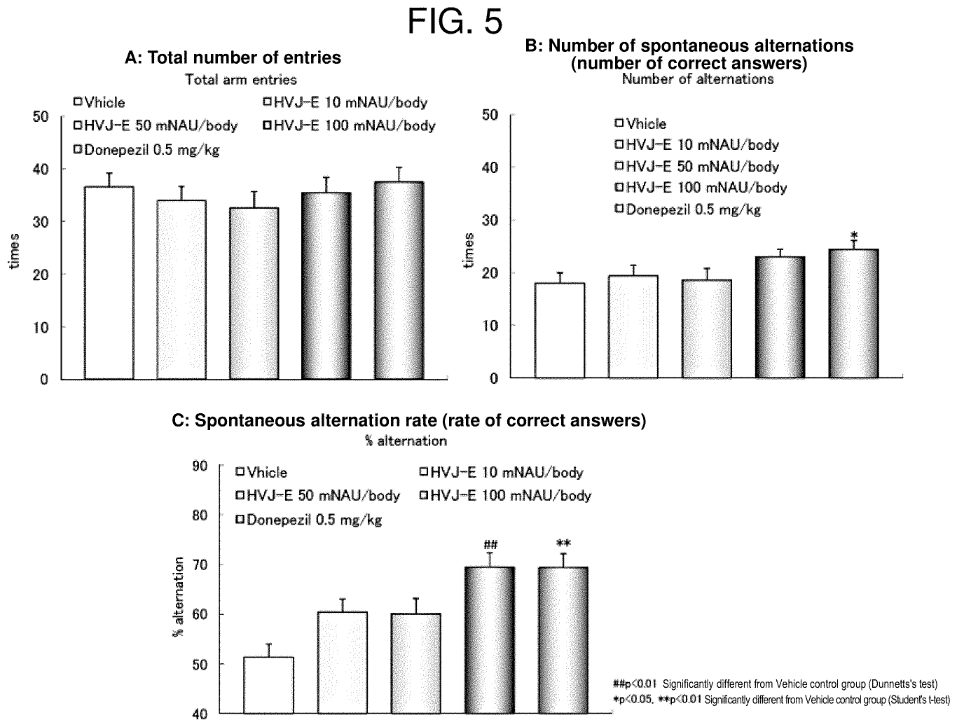

The result of the Y-maze test is shown in FIG. 5. Similar to the result in Example 2, the HVJ-E (100 mNAU/body) intranasal administration group exhibited a significantly higher spontaneous alternation rate (P<0.01) in comparison with the vehicle control group and the numerical value was equivalent to the donepezil group.

The t-test between 2 groups on the HVJ-E administration group and the Vehicle group provided p values of 0.0204 and 0.0515 respectively for 50 mNAU/body and 10 mNAU/body, indicating significant difference for 50 mNAU/body and also the same tendency for 10 mNAU/body (the p value for 100 mNAU/body was 0.0000368). From the foregoing, the dose dependent effect of the HVJ-E single intranasal administration was confirmed. Furthermore, the effect of HVJ-E is expected to be increased by optimizing the dose and the route of administration.

### INDUSTRIAL AVAILABILITY

The present invention is useful for prevention and/or treatment of cognitive impairment and/or neurodegenerative diseases with accumulation of a prionoid.

All publications, patents, and patent applications cited herein are incorporated herein by reference as they are.

1.-11. (canceled)

12. A method for preventing and/or treating cognitive impairment and/or a neurodegenerative disease with accumulation of a prionoid, the method comprising administering a Sendai virus envelope to a patient in need thereof.

13. The method according to claim 12, wherein the Sendai virus envelope is a wild type Sendai virus envelope.

14. The method according to claim 12, wherein the Sendai virus envelope is an inactivated Sendai virus envelope.

15. The method according to claim 12, wherein the Sendai virus envelope is an inactivated wild type Sendai virus envelope.

16. The method according to claim 12, wherein the cognitive impairment and/or a neurodegenerative disease with accumulation of a prionoid is any one selected from Alzheimer-type dementia, mild cognitive impairment (MCI), cerebral amyloid angiopathy, Down syndrome, macular degeneration, dementia with Lewy bodies, Parkinson's disease, multiple system atrophy, tauopathy, frontotemporal lobar degeneration, argyrophilic grain dementia, amyotrophic lateral sclerosis, autism, diabetes, amyotrophic lateral sclerosis (ALS), and Creutzfeldt-Jakob disease.

17. The method according to claim 16, wherein the cognitive impairment and/or a neurodegenerative disease with accumulation of a prionoid is Alzheimer-type dementia.

18. The method according to claim 12, wherein the prionoid is amyloid (3.

19. The method according to claim 12, wherein the Sendai virus envelope is administered in combination with an immune checkpoint inhibitor.

20. The method according to claim 19, wherein the Sendai virus envelope and the immune checkpoint inhibitor are administered simultaneously or sequentially.

21. The method according to claim 19, wherein the Sendai virus envelope and the immune checkpoint inhibitor are provided as a combination drug, a combined administration of separate agents, or a kit.

22. The method according to claim 19, wherein the immune checkpoint inhibitor is any one or more selected from the group consisting of an anti-PD-1 antibody, an anti-PD-L1 antibody, an anti-PD-L2 antibody, an anti-CTLA-4 antibody, an anti-MR antibody, an anti-CD137 antibody, an anti-LAG-3 antibody, an anti-OX40 antibody, an anti-CD80 antibody, an anti-CD86 antibody, an anti-B7-H3 antibody, an anti-B7-H4 antibody, an anti-B7-H5 antibody, an anti-TIM-3 antibody, an anti-TIGIT antibody, and an anti-BTLA antibody.

23. The method according to claim 22, wherein the immune checkpoint inhibitor is an anti-PD-1 antibody, an anti-PD-L1 antibody, or an anti-CTLA-4 antibody.
